(19) United States
(12) Patent Application Publication
(10) Pub. No.: US 2024/0032677 A1
(43) Pub. Date: Feb. 1, 2024

(54) AROMATHERAPY WATER CUP

(71) Applicant: WaterH Inc., Toronto (CA)

(72) Inventors: XIJIN WU, SHENZHEN CITY (CN); XIA QU, SHENZHEN CITY (CN); QI WANG, SHENZHEN CITY (CN); YALU MA, SHENZHEN CITY (CN)

(21) Appl. No.: 17/876,646

(22) Filed: Jul. 29, 2022

(51) Int. Cl. A45F 3/18; A61L 9/12

(52) U.S. Cl. CPC A45F 3/18; A61L 9/12

(57) ABSTRACT

An aromatherapy water cup relates to the technical field of drinking utensils, comprising a cup body and a cover body covered thereon, wherein a water absorbing portion is provided on the cover body, an installation portion is provided on a side of the cover body close to the drawn water portion, the installation portion is used for detachably assembling an aromatherapy box, and a plurality of ventilation holes are provided on the aromatherapy box. The installation portion is an installation groove, a limit snap seat is arranged on the installation groove, and the aromatherapy box is correspondingly provided with a snap-fit slot for snap-fit with the limit snap seat. The limit snap seat is arranged on a vertical centerline of the installation groove, and the aromatherapy box is arranged in a ring shape and is sleeved on the limit snap seat.

### FIELD OF THE DISCLOSURE

The present disclosure relates to the technical field of drinking utensils, in particular, to an aromatherapy water cup.

### BACKGROUND OF THE DISCLOSURE

As a daily necessity, drinking glasses are widely used in home and office, and currently popular aromatherapy products are also widely used in homes and offices to remove odors or refresh the mind. However, the current aromatherapy products are not combined with water cup products, and aromatherapy products are only suitable for occasions that are used in a fixed position for a long time, so as to generate enough fragrance atmospheres, which is inconvenient to carry around, and cannot quickly emit enough fragrance in different places.

At present, there are many kinds of water cups, but there are no related products and technologies with aromatherapy function announced, so that people can use the water cups on the move, and in addition, they can quickly and intuitively feel the fragrance atmosphere, refresh the mind or remove peculiar smells with their breath anytime, anywhere.

### SUMMARY OF THE DISCLOSURE

The purpose of the present disclosure is to overcome the defects and deficiencies of the prior art to provide an aromatherapy water cup, which enables the user to quickly and intuitively feel the fragrance atmosphere, refresh the mind or remove peculiar smell with his nose anytime, anywhere while constantly moving and using the water cup.

For achieving the above object, the technical scheme adopted in the present disclosure is to provide an aromatherapy water cup, comprising a cup body and a cover body covered thereon, wherein a water absorbing portion is provided on the cover body, an installation portion is provided on a side of the cover body close to the drawn water portion, the installation portion is used for detachably assembling an aromatherapy box, and a plurality of ventilation holes are provided on the aromatherapy box.

The installation portion is an installation groove, a limit snap seat is arranged on the installation groove, and the aromatherapy box is correspondingly provided with a snap-fit slot for snap-fit with the limit snap seat.

The limit snap seat is arranged on a vertical centerline of the installation groove, and the aromatherapy box is arranged in a ring shape and is sleeved on the limit snap seat.

The water absorbing portion is arranged on the limit snap seat, and passes through an inner ring of the aromatherapy box.

The water absorbing portion is a suction nozzle, a bottom of the suction nozzle is connected with a suction pipe, one end of the suction pipe is fixed at a bottom of the cover body, and another end of the suction pipe extends inside the cup body.

A protrusion is further provided on a side surface of the limit snap seat, the snap-fit slot is correspondingly provided with a groove, and the protrusion cooperates with the groove to fix the aromatherapy box.

The cover body is further provided with a top cover, the top cover is hinged with the cover body, and the cover body covers the aromatherapy box and the water absorbing portion.

A press-to-open assembly is further provided between the cover body and the top cover, the press-to-open assembly is used for the cover body to lock with the top cover, and after pressing the press-to-open assembly, the cover body and the top cover are released from a locked state.

An upper half of the cup body is curved, and a curved angle is to 35°.

An anti-skid structure is provided on the cup body.

After adopting the above-mentioned technical solution, the beneficial effects of the present disclosure are as follows.

1. The body of the cup is provided with a mouth, a cover is arranged on the top of the mouth, and a water absorbing portion is arranged on the cover. The water absorbing portion is used to absorb the liquid inside the cup through the mouth of the cup when the user sucks it to the user's mouth. There is also an aromatherapy box on the side close to the water absorbing portion of the lift. The aromatherapy box can be placed in the aromatherapy box. The aromatherapy box is provided with a plurality of ventilation holes, so that fragrances or other fragrance substances can be emitted to the surrounding environment through the ventilation holes.

2. The cover body is also provided with a top cover, the top cover is hinged with the cover body, and the cover body is covered with an aromatherapy box and a water absorbing portion, so as to effectively prevent external unclean substances from entering the aromatherapy box or the water absorbing portion. There is also a press-to-open assembly between the cover body and the top cover. The press-to-open assembly is used to lock the cover body and the top cover when the top cover is placed on the cover body, and after the user presses the rotary plate on it, the press-to-open assembly can be used for locking, so that the cover body and the top cover are released from the locked state, and the top cover can be opened at this time.

### BRIEF DESCRIPTION OF THE DRAWINGS

In order to illustrate the embodiments of the present invention or the technical solutions in the prior art more clearly, the following briefly introduces the accompanying drawings that are used in the description of the embodiments or the prior art. Obviously, the drawings in the following description are only are some embodiments of the present disclosure. For those of ordinary skill in the art, other drawings can also be obtained based on these drawings without any creative effort.

FIG. 1 is a schematic diagram of the structure of the present disclosure.

FIG. 2 is an exploded diagram corresponding to FIG. 1.

FIG. 3 is a schematic diagram of the cover body of the present disclosure.

FIG. 4 is an exploded diagram of the first part of the structure corresponding to FIG. 1.

FIG. 5 is an exploded diagram of the second part of the structure corresponding to FIG. 1.

Reference numeral: 1. cup body; 11. cup mouth; 2. cover body; 21. limit snap seat; 211. protrusion; 22. rope loop; 23. rotating shaft; 24. installation portion; 25. suction nozzle; 26. aromatherapy box; 261. snap slot; 27. snap cover; 271. snap ring; 28. through hole; 3. top cover; 31. axle seat; 4. press-to-open assembly; 41. rotating plate; 411. buckle; 42. spring; 43. accommodating cavity; 44. clamping block.

### DETAILED DESCRIPTION OF THE EXEMPLARY EMBODIMENTS

The present disclosure will be further described in detail below with reference to the accompanying drawings.

This specific embodiment is only an explanation of the present disclosure, and it is not a limitation of the present disclosure. Those skilled in the art can make modifications to this embodiment without creative contribution as needed after reading this specification, and as long as they are within the scope of the claims of the present disclosure, they are protected by the patent law.

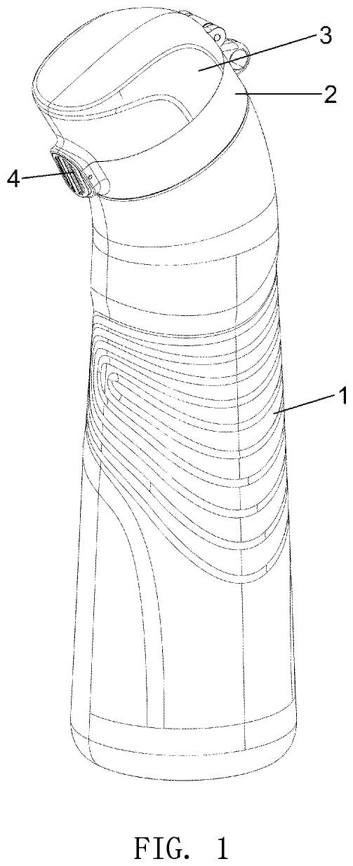

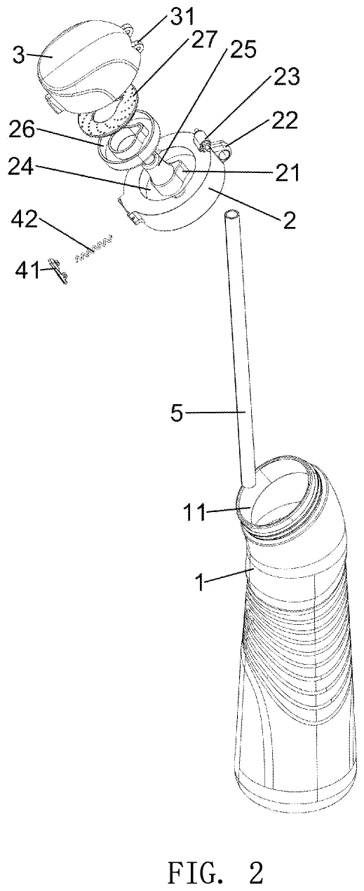

The present embodiment relates to an aromatherapy water cup, as shown in FIG. 1 and FIG. 2.

The cup body 1 is provided with a cup mouth 11, the cup mouth 11 is provided with an external thread, the cup mouth 11 is covered with a cover body 2, and the lower part of the cover body 2 is provided with an inner thread that is assembled with the outer thread. The cup body 1 and the cover body 2 are assembled by thread to improve the sealing and firmness. When the user is sucking, the liquid inside the cup body 1 is sucked into the user's mouth through the cup mouth 11.

What needs to be added is that there is also an aromatherapy box 26 on the side close to the water absorbing portion of the lift. The aromatherapy box 26 can be placed in it for spices or other fragrance substances. The fragrance of perfume or other fragrance substances can be emitted to the surrounding environment through the ventilation holes.

What needs to be added is that the aromatherapy box 26 is composed of a box body and a snap cover 27, the box body is placed in which the fragrance or other fragrance substances are placed, and a snap ring 271 is provided at the bottom of the snap cover 27. The circumference of the snap ring 271 is smaller than that of the snap cover 27. The snap ring 271 is used to snap on the opening of the box body, and the ventilation hole is also provided on the snap cover 27.

Preferably, the installation portion 24 is an installation groove, the installation groove is opened in a cylindrical shape, and a limit snap seat 21 is arranged on the vertical center line of the installation groove, and the limit snap seat 21 is arranged in a rod shape, and the top surface of the rod is connected to the cover body. The upper plane of the cover body 2 is flush, and the aromatherapy box 26 is correspondingly provided with a snap slot 261 that is snapped with the limit snap seat 21. The snap slot 261 in the middle of the annular aromatherapy box 26 is inserted into the limit snap seat 21, and at the same time, the box body of the aromatherapy box 26 is fitted into the installation groove, so as to embody the assembly of the aromatherapy box 26.

What needs to be added is that the water absorbing portion is arranged on the limit snap seat 21, and the snap slot 261 in the middle of the aromatherapy box 26 is penetrated, so that fragrance is distributed around the water supply portion, and the fragrance effect is better when drinking water. The water absorbing portion is a suction nozzle 25. The bottom of the suction nozzle 25 is provided with a through hole 28. The through hole 28 is used for the suction pipe to be clamped on it. One end of the suction pipe is fixed at the bottom of the cover body 2, and the other end extends inside the cup body 1. When the user sucks through the suction nozzle 25, the suction nozzle 25 sucks the liquid in the cup body 1 into the user's mouth through the suction tube. Both the straw and the water absorbing portion are made of PP material to improve heat resistance.

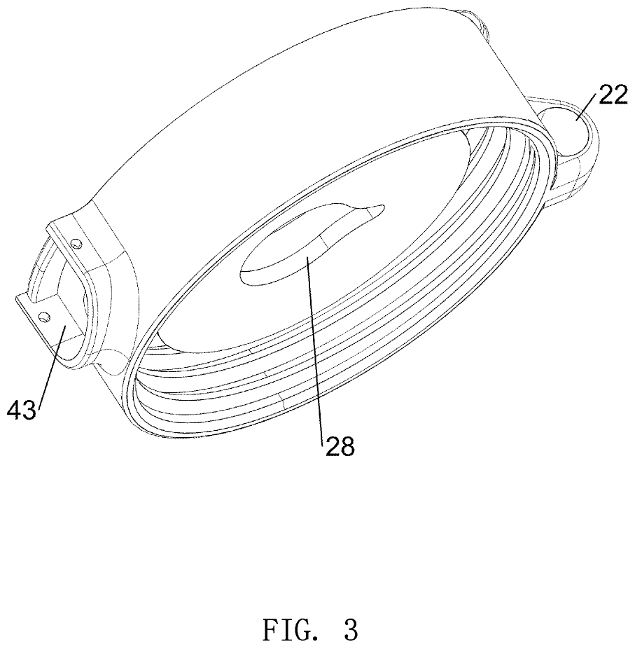

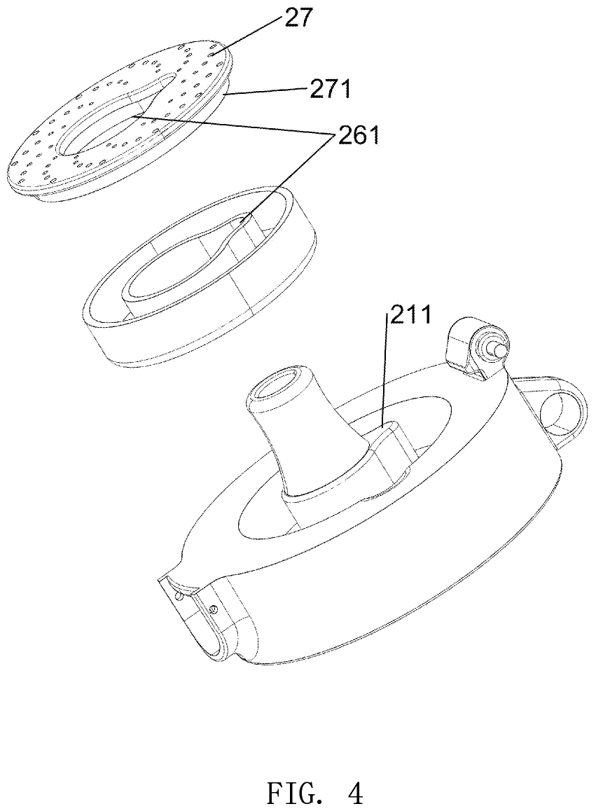

Preferably, as shown in FIG. 3 and FIG. 4, a protrusion 211 is further provided on the side of the limit snap seat 21, the protrusion 211 occupies a part of the installation groove from the side direction. The snap groove 261 is also provided with a corresponding groove, and the protrusion 211 cooperates with the groove to fix the aromatherapy box 26, thereby preventing the aromatherapy box 26 from rotating in the installation groove so as to play a positioning role.

Preferably, the side surface of the cover body 2 is provided with a rope loop 22, and the rope loop 22 is integrally formed with the cover body 2. The rope loop 22 is used for the rope to pass through, so that the rope can be passed through, which is convenient for carrying the device.

Preferably, the cover body 2 is also provided with a top cover 3, the top cover 3 is hinged with the cover body 2, and the cover body 2 is covered with an aromatherapy box 26 and a water absorbing portion, thereby effectively preventing external unclean substances from entering the aromatherapy box 26 or the water absorbing portion. The cover body 2 is provided with a rotating shaft 23. Correspondingly, the top cover 3 is provided with a shaft seat 31, and the shaft seat 31 is assembled with the rotating shaft 23, so that the top cover 3 and the cover body 2 can be hingedly connected.

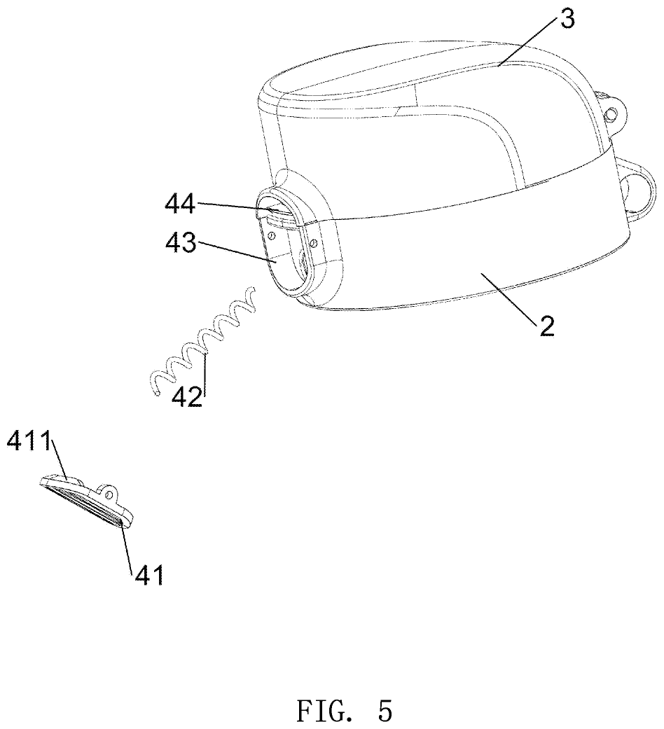

As shown in FIG. 5, a press-to-open assembly 4 is provided between the cover body 2 and the top cover 3. The press-to-open assembly 4 is arranged on the end of the cover body 2 and the top cover 3 away from the hinge portion. The push-to-open assembly 4 is used to lock the top cover 3 when the top cover 3 is placed on the cover body 2, and after the user presses the rotating plate 41 on it, the cover body 2 and the top cover 3 can be unlocked while the top cover 3 can be opened.

It should be added that the press-to-open assembly 4 includes: an accommodating cavity 43 provided on a side surface of the cover body 2. The accommodating cavity 43 is located at one end away from the hinge portion, the accommodating cavity 43 is provided with a rotating plate 41, the central axis of rotation of the rotating plate 41 is located near the top of the accommodating cavity 43, a part of the rotating plate 41 is covered with the accommodating cavity 43, and the other part is provided with a buckle 411. The top cover 3 is also provided with a matching groove, the rotating plate 41 can cover the matching groove through another part, and the clamping block 44 is arranged in the matching groove to be engaged with the buckle 411. A spring 42 is connected between the accommodating cavity 43 and the lower part of the rotating plate 41. When the lower part of the rotating plate 41 is pressed, the lower part of the rotating plate 41 sinks into the accommodating cavity 43, and the upper part of the rotating plate 41 is lifted accordingly, and at this time, the buckle 411 is disengaged from the clamping block 44 to embody the unlocking of the top cover 3 and the cover body 2. When the lower part of the rotating plate 41 is loosened, the spring 42 is used to reset it, and when the top cover 3 is closed, the snap connection to the top cover 3 can be embodied again.

Preferably, the upper part of the cup body 1 is curved, and its curved angle is 15° to 35°. The curved upper part of the water cup will make the cover body 2 also incline, so that the suction nozzle 25 on the cover body 2 is also inclined accordingly. The inclination is convenient for the user to suck the suction nozzle 25. The cup body 1 is provided with an anti-skid structure, so as to improve the frictional force when holding the water cup, so that the holding of the water cup is firmer and prevents falling.

The above is only used to illustrate the technical solution of the present disclosure and not to limit it. Regarding other modifications or equivalent replacements made by those of ordinary skill in the art to the technical solution of the present disclosure, as long as they do not depart from the spirit and scope of the technical solution of the present disclosure, they should be included in the within the scope of the claims of the present disclosure.

What is claimed is:

1. An aromatherapy water cup, comprising a cup body and a cover body covered thereon, wherein a water drawing portion is provided on the cover body, an installation portion is provided on a side of the cover body close to the water drawing portion, the installation portion is used for detachably assembling an aromatherapy box, and a plurality of ventilation holes are provided on the aromatherapy box.

2. The aromatherapy water cup according to claim 1, wherein the installation portion is an installation groove, a limit snap seat is arranged on the installation groove, and the aromatherapy box is correspondingly provided with a snap-fit slot for snap-fit with the limit snap seat.

3. The aromatherapy water cup according to claim 2, wherein the limit snap seat is arranged on a vertical centerline of the installation groove, and the aromatherapy box is arranged in a ring shape and is sleeved on the limit snap seat.

4. The aromatherapy water cup according to claim 3, wherein the water drawing portion is arranged on the limit snap seat, and passes through an inner ring of the aromatherapy box.

5. The aromatherapy water cup according to claim 4, wherein the water drawing portion is a suction nozzle, a bottom of the suction nozzle is connected with a suction pipe, one end of the suction pipe is fixed at a bottom of the cover body, and another end of the suction pipe extends inside the cup body.

6. The aromatherapy water cup according to claim 3, wherein a protrusion is further provided on a side surface of the limit snap seat, the snap-fit slot is correspondingly provided with a groove, and the protrusion cooperates with the groove to fix the aromatherapy box.

7. The aromatherapy water cup according to claim 1, wherein the cover body is further provided with a top cover, the top cover is hinged with the cover body, and the cover body covers the aromatherapy box and the water drawing portion.

8. The aromatherapy water cup according to claim 7, wherein a press-to-open assembly is further provided between the cover body and the top cover, the press-to-open assembly is used for the cover body to lock with the top cover, and after pressing the press-to-open assembly, the cover body and the top cover are released from a locked state.

9. The aromatherapy water cup according to claim 1, wherein an upper half of the cup body is curved, and a curved angle is 15° to 35°.

10. The aromatherapy water cup according to claim 1, wherein an anti-skid structure is provided on the cup body.
